(19) United States
(12) Patent Application Publication
(10) Pub. No.: US 2005/0240166 A1
(43) Pub. Date: Oct. 27, 2005

(54) Implantable device, formulation and method for anti-psychotic therapy using risperidone

(75) Inventors: Derek J. Harper, Goleta, CA (US); Charles F. Milo, Los Altos, CA (US)

Correspondence Address: YOUNG LAW FIRM;A PROFESSIONAL CORPORATION, 4370 ALPINE ROAD SUITE 106, PORTOLA VALLEY, CA, 94028, US

(73) Assignee: Microsolutions, Inc., Goleta, CA (US)

(21) Appl. No.: 11/112,331

(22) Filed: Apr. 22, 2005

Related U.S. Application Data
(60) Provisional application No. 60/565,712, filed on Apr. 26, 2004.

(51) Int. Cl. A61K 31/519; A61K 9/22

(52) U.S. Cl. 604/890.1; 514/259.41

(57) ABSTRACT

An subcutaneously implantable device for delivering Risperidone to a patient includes a pump, a compartment configured to store a pharmaceutical formulation, and a volume of the pharmaceutical formulation loaded in the compartment, the pharmaceutical formulation including Risperidone solvated or suspended in a pharmaceutically acceptable solvent in concentration of at least 50 milligrams per milliliter. The implanted pump may then deliver a therapeutically effective dose of Risperidone to the patient.

### CROSS-REFERENCE TO RELATED APPLICATIONS

This application claims priority under 35 U.S.C. § 119(e) to provisional application Ser. No. 60/565,712, filed Apr. 26, 2004, which application is hereby incorporated by reference in its entirety.

This document discloses an implantable device, drug formulation and method of anti-psychotic therapy using a Risperidone formulation. The Risperidone formulation may be used for many types of pumps; both osmotic, and other pump technologies. Advantageously, however, the Risperidone formulation described herein may be readily used in conjunction with the osmotic pumps described and claimed in commonly assigned U.S. Pat. No. 6,436,091, entitled “Methods and Implantable Devices and Systems for Long Term Delivery of a Pharmaceutical Agent”, U.S. Pat. No. 6,616,652, entitled “Osmotic Pump Delivery System with Pre-Hydrated Membrane(s) and/or Primable Catheter”, U.S. Pat. No. 6,632,217, entitled “Implantable Osmotic Pump”, and in commonly assigned and copending U.S. patent application Ser. No. 10/386,919, filed Mar. 11, 2003, entitled “Implantable Devices with Invasive and Non-Invasive Reversible Infusion Rate Adjustability” (hereinafter MICR5701 CIP), the disclosures of each hereby incorporated herein by reference in their entirety.

### BACKGROUND OF THE INVENTION

The treatment of schizophrenic episodes and other psychotic conditions, in which positive symptoms (such as hallucinations, delusions, thought disturbances, hostility, suspiciousness) and/or negative symptoms (such as blunted affect, emotional and social withdrawal, poverty of speech) are prominent. Schizophrenia is the most common form of severe mental illness, with a lifetime risk of developing the disease of 0.5-1%. It is a disorder that affects a person's reasoning and thought processes, emotions and behavior, which may be severe and disabling.

The treatment of schizophrenia and psychotic conditions using orally administered Risperidone (Risperdal™) is well established; however patient compliance with the oral administration is a significant issue which causes re-lapse (or under-treatment) in a large number of the afflicted patient population. Commonly prescribed oral anti-psychotics include Risperidone, Olanzapine and Amisulpride. The typical oral dose of Risperidone is 0.25-16 milligrams per day and the most common oral dose is 2-4 milligrams per day.

Another treatment option for schizophrenia is the use of longer-acting depot anti-psychotic injections. These injections are used for maintenance therapy especially when compliance with oral administration is unreliable. Depots of Flupentixol, Fluphenazine, Haloperidol, Zuclopenthixol, and Pipotiazine are available. They are administered by deep intra-muscular (IM) injection, repeated at intervals ranging from 14-35 days.

Recently, Risperidone has been approved for IM administration as Risperdal Consta™, which is an injectable suspension of Risperidone. Risperdal Consta is administered by deep IM glutal injection every two (2) weeks. In patients with no previous history of Risperidone use, oral Risperidone is used initially in order to access the required dose and tolerability prior to commencing IM treatment. The disadvantages of depot medications and parenteral injections are that some patients are reluctant to accept repeated injections, and if adverse effects occur they may be difficult to manage because of the inability to rapidly discontinue the medication. Another disadvantage is that even for depot injections, the patient must return every two to four (2-4) weeks for another injections, which still raises concerns about patient compliance with the treatment.

### DETAILED DESCRIPTION

It is an objective of the invention to provide a pharmaceutically acceptable formulation containing Risperidone for continuous subcutaneous infusion in a patient.

It is another objective of this invention to provide an osmotic pump for implantation in a psychotic patient and for the pump to continuously deliver the Risperidone formulation to the patient. The pump is preferably implanted subcutaneously in the patient and may be designed to deliver Risperidone in a therapeutically effective range from 1 month up to several years; however there is a tradeoff between physical size of the pump and the duration of therapy. For example, one preferred pump design is a 1 milliliter volume discoid pump intended to continuously deliver Risperidone for approximately 6-12 months as described further below. The pump may be implanted subcutaneously in the inner aspect of the upper arm, if the pump is configured in a cylindrical geometry, such as described in U.S. Pat. Nos. 5,728,396 and 6,689,373 or U.S. Pat. Nos. 6,436,091 and 6,616,652. The pump may be advantageously implanted subcutaneously in the upper chest wall, if the pump is configured in a discoid geometry, such as described in U.S. Pat. No. 6,632,217, and MICR5701CIP. The pump may be implanted subcutaneously in other regions of the anatomy depending of the size and geometry of the pump.

It is yet another objective of this invention to provide an adjustable flow-rate osmotic pump for delivery of Risperidone. In one embodiment the flow-rate of the pump may be selected within a predetermined range prior to implantation. In another embodiment the flow-rate of the pump may be selected within a predetermined range after implantation by a percutaneous adjustment method. In yet another embodiment of the invention, the flow-rate of the pump may be non-invasively adjusted within a predetermined range.

### BRIEF DESCRIPTION OF THE DRAWINGS

FIG. 1 shows a perspective view of an implantable pump system suitable for use with embodiments of the present invention.

FIG. 2 is a cross sectional view of an implantable osmotic pump suitable for use in conjunction with embodiments of the present invention, showing the pharmaceutical agent compartment.

### BRIEF DESCRIPTION OF THE DRAWINGS

According to an embodiment thereof, the present invention is an implantable device for delivering Risperidone to a patient, including a pump; a compartment configured to store a pharmaceutical formulation; a volume of the pharmaceutical formulation loaded in the compartment, the pharmaceutical formulation including Risperidone solvated or suspended in a pharmaceutically acceptable solvent in concentration of at least 50 milligrams per milliliter.

The pump may be an osmotic pump. The pump may be a flow-rate adjustable osmotic pump. The pharmaceutically acceptable solvent may include Benzyl Alcohol. The pharmaceutically acceptable solvent may include Benzyl Benzoate. The pharmaceutical formulation may contain stabilizers, antioxidants, surfactants and/or emulsifiers. The pharmaceutical formulation may contain a Risperidone congener, derivative and/or salt.

According to another embodiment thereof, the present invention is an implantable device for delivering Risperidone to a patient, including a pump; a compartment adapted to store a pharmaceutical formulation; a volume of the pharmaceutical formulation loaded in the compartment, wherein the pharmaceutical formulation comprises a paste and/or gel containing suspended Risperidone in a concentration of at least 50 milligrams per milliliter. The pump may be an osmotic pump. The pump may be a flow-rate adjustable osmotic pump. The pharmaceutical formulation may contain a Risperidone congener, derivative and/or salt.

Another embodiment of the present invention is a method of providing anti-psychotic treatment to a patient, including steps of providing an implantable pump loaded with a Risperidone formulation; implanting the pump subcutaneously in the patient, the pump delivering a therapeutically effective dose of Risperidone to the patient.

The pump may be an osmotic pump. The pump may be a flow-rate adjustable osmotic pump.

### DETAILED DESCRIPTION OF THE INVENTION

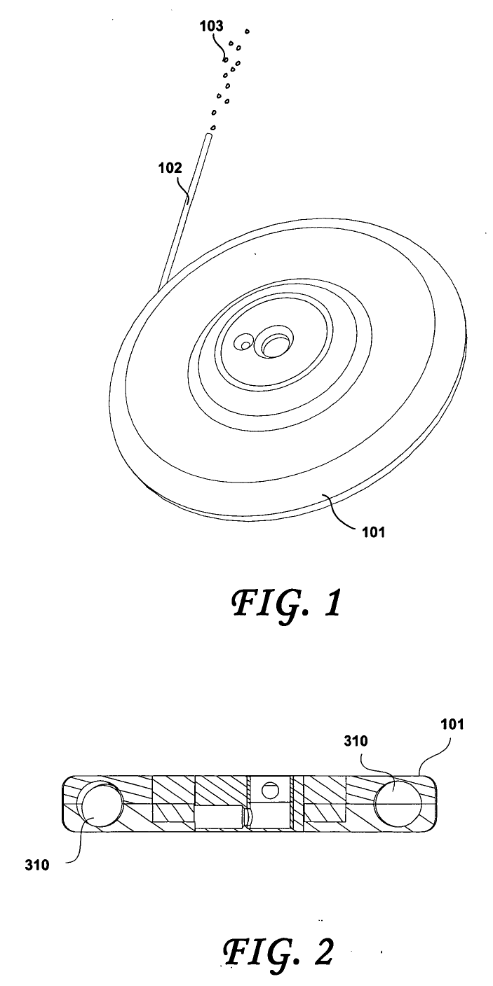

An exemplary pump suitable for use with the present invention is shown in FIG. 1. The pump 101 may be fitted with a catheter 102 to dispense a Risperidone formulation 103 according to one of the embodiments disclosed herein. In one embodiment, a Risperidone formulation may be prepared by adding 1.2 grams of Benzyl Alcohol (available from Spectrum Chemical, Gardena, Calif., USA, for example) to 845 milligrams of Risperidone (available from TEVA Ltd. A.P.I. Division, Petah Piqva, Israel, for example) in a vial. The sealed vial with solution may be placed in an ultra-sonic bath to completely dissolve the Risperidone. The resulting solution is clear and approximately 1.8 milliliters in volume, yielding a concentration of approximately 470 milligrams Risperidone per milliliter (0.47 milligrams per microliter). Alternatively, Benzyl Benzoate may be chosen as the solvent for Risperidone. The formulation may be based on a suitable Risperidone congener, derivative, and/or salt; and the formulation may contain stabilizers, antioxidants, surfactants and/or emulsifiers. The Risperidone formulation may be loaded in the drug compartment 310 of an osmotic pump 101 shown in cross section in FIG. 2, such as the pump described in U.S. Pat. No. 6,632,217, for example. The pump may be designed to deliver the formulation at either of the following rates: 2.0, 4.0, and 6.0 microliters/day, for example. Considering that such a pump loaded with 1000 microliters of formulation containing 0.47 milligrams Risperidone per microliter, the delivery of Risperidone from the pump is either 0.94, 1.88, or 2.82 milligrams per day for approximately 500 days, 250 days or 170 days; respectively. The absolute oral bioavailability of orally delivered Risperidone is 0.7; therefore, the 0.94, 1.88, and 2.82 mg/day dosages would convert to 1.34, 2.69, and 4.02 milligrams per day of orally delivered Risperidone. If desired, the flow-rate of the pump may be selected prior to implantation or adjusted up after implantation as described in U.S. Pat. No. 6,632,217; or alternatively the flow-rate of the pump may be constant throughout the duration of therapy.

In another embodiment, the previously described Risperidone formulation may be loaded in an osmotic pump such as the pump described in co-pending MICR5701CIP and the pump designed to deliver the formulation at either of the following exemplary rates: 2.0, 4.0, and 6.0 microliters/day, resulting in a delivery of Risperidone from the pump of either 0.94, 1.88, or 2.82 milligrams per day for 500 days, 250 days or 160 days; respectively. If desired, the flow-rate of the pump may be adjusted up or down after implantation as described in MICR5701CIP; or alternatively the flow-rate of the pump may be constant throughout the duration of therapy.

In yet another alternative embodiment, a Risperidone formulation may be prepared comprising micronized particles of Risperidone suspended in a gel carrier. The micronized Risperidone may be prepared using a mortar and pistil and the resulting powder sifted to produce particles with a diameter <50 microns. A paste formulation of Risperidone may then be prepared by adding 1.2 grams of micronized Risperidone to 1.8 grams of Glycerin (available from Sigma-Aldrich Chemical, for example) and adding 1% Sodium Dodecyl Sulphate (also available from Sigma-Aldrich Chemical, for example) to aid in suspending the particles in the paste. The micronized Risperidone and gel may then be mechanically stirred and mixed until the particles are suspended substantially evenly in the gel carrier. The resulting paste is approximately 2.4 milliliters in volume, yielding a concentration of approximately 500 milligrams Risperidone per milliliter. The formulation may be based on a suitable Risperidone congener, derivative, and/or salt; and the formulation may contain stabilizers, antioxidants, surfactants and/or emulsifiers. This paste Risperidone formulation may be loaded in an osmotic pump such as the pump described (for example) in U.S. Pat. No. 6,632,217 and/or co-pending MICR5701CIP; and the pump designed to infuse the formulation at the aforementioned exemplary delivery rates and duration of therapy. In general, it is desirable to configure the pump and formulation to infuse therapeutic dosages. For Risperidone, therapeutic dosages may range from 0.01 mg/day to 20 mg/day, and the preferred delivery longevity ranges from 14 days to 2 years. More preferably, the desired dosages range from 0.25 mg/day to 6 mg/day for periods ranging from 60 days to 1 year.

Alternatively the Risperidone formulations described above may be loaded in an osmotic pump such as the pump described in U.S. Pat. Nos. 5,728,396 and 6,689,373; and the flow-rate of the pump may be designed to deliver a therapeutically effective dose of Risperidone when the pump is implanted subcutaneously in the inner aspect of a patient's upper arm such as described in these patents for delivery of other medications. then be prepared by adding 1.2 grams of micronized Risperidone to 1.8 grams of Glycerin (available from Sigma-Aldrich Chemical, for example) and adding 1% Sodium Dodecyl Sulphate (also available from Sigma-Aldrich Chemical, for example) to aid in suspending the particles in the paste. The micronized Risperidone and gel may then be mechanically stirred and mixed until the particles are suspended substantially evenly in the gel carrier. The resulting paste is approximately 2.4 milliliters in volume, yielding a concentration of approximately 500 milligrams Risperidone per milliliter. The formulation may be based on a suitable Risperidone congener, derivative, and/or salt; and the formulation may contain stabilizers, antioxidants, surfactants and/or emulsifiers. This paste Risperidone formulation may be loaded in an osmotic pump such as the pump described (for example) in U.S. Pat. No. 6,632,217 and/or co-pending MICR5701CIP; and the pump designed to infuse the formulation at the aforementioned exemplary delivery rates and duration of therapy. In general, it is desirable to configure the pump and formulation to infuse therapeutic dosages. For Risperidone, therapeutic dosages may range from 0.01 mg/day to 20 mg/day, and the preferred delivery longevity ranges from 14 days to 2 years. More preferably, the desired dosages range from 0.25 mg/day to 6 mg/day for periods ranging from 60 days to 1 year.

Alternatively the Risperidone formulations described above may be loaded in an osmotic pump such as the pump described in U.S. Pat. Nos. 5,728,396 and 6,689,373; and the flow-rate of the pump may be designed to deliver a therapeutically effective dose of Risperidone when the pump is implanted subcutaneously in the inner aspect of a patient's upper arm such as described in these patents for delivery of other medications.

1. An implantable device for delivering Risperidone to a patient, comprising:
a pump;
a compartment configured to store a pharmaceutical formulation;
a volume of the pharmaceutical formulation loaded in the compartment, the pharmaceutical formulation including Risperidone solvated or suspended in a pharmaceutically acceptable solvent in concentration of at least 50 milligrams per milliliter.

2. The device of claim 1, wherein the pump is an osmotic pump.

3. The device of claim 1, wherein the pump is a flow-rate adjustable osmotic pump.

4. The device of claim 1, wherein the pharmaceutically acceptable solvent includes Benzyl Alcohol.

5. The device of claim 1, wherein the pharmaceutically acceptable solvent includes Benzyl Benzoate.

6. The device of claim 1, wherein the pharmaceutical formulation contains stabilizers, antioxidants, surfactants and/or emulsifiers.

7. The device of claim 1, wherein the pharmaceutical formulation contains a Risperidone congener, derivative and/or salt.

8. An implantable device for delivering Risperidone to a patient, comprising:
a pump;
a compartment adapted to store a pharmaceutical formulation;
a volume of the pharmaceutical formulation loaded in the compartment, wherein the pharmaceutical formulation comprises a paste and/or gel containing suspended Risperidone in a concentration of at least 50 milligrams per milliliter.

9. The device of claim 8, wherein the pump is an osmotic pump.

10. The device of claim 8, wherein the pump is a flow-rate adjustable osmotic pump.

11. The device of claim 8, wherein the pharmaceutical formulation contains a Risperidone congener, derivative and/or salt.

12. A method of providing anti-psychotic treatment to a patient, comprising:
providing an implantable pump loaded with a Risperidone formulation;
implanting the pump subcutaneously in the patient;
the pump delivering a therapeutically effective dose of Risperidone to the patient.

13. The method of claim 8, wherein the pump is an osmotic pump.

14. The method of claim 8, wherein the pump is a flow-rate adjustable osmotic pump.
